(19) United States
(12) Patent Application Publication
(10) Pub. No.: US 2022/0256888 A1
(43) Pub. Date: Aug. 18, 2022

(54) COMPOSITIONS AND METHODS USING AT LEAST ONE OF OLEUROPEIN OR A METABOLITE THEREOF TO TREAT OR PREVENT MUSCLE FATIGUE FROM EXERCISE AND/OR FOR RESISTANCE TO MUSCLE FATIGUE FROM EXERCISE

(71) Applicant: SOCIÉTÉ DES PRODUITS NESTLÉ S.A., Vevey (CH)

(72) Inventors: Umberto De Marchi, Le Lignon (CH); Marie Noelle Horcajada, Echenevex (FR); Jerome Feige, Crissier (CH)

(21) Appl. No.: 17/736,496

(22) Filed: May 4, 2022

Related U.S. Application Data
(63) Continuation-in-part of application No. 17/595,166, filed on Nov. 10, 2021.
(60) Provisional application No. 62/847,076, filed on May 13, 2019.

(51) Int. Cl. A23K 20/121; A23K 20/111; A23L 33/105; A23L 33/16; A23L 33/00; A61K 31/05; A61K 31/351; A61K 31/7048; A61K 33/06; A61P 21/00

(52) U.S. Cl. CPC A23K 20/121; A23K 20/111; A23L 33/105; A23L 33/16; A61P 21/00; A61K 31/05; A61K 31/351; A61K 31/7048; A61K 33/06; A23L 33/40

(57) ABSTRACT

A method of preventing or treating muscle fatigue from exercise and/or for resistance to muscle fatigue from exercise, the method including orally administering at least one of oleuropein or metabolite thereof to an individual before, during and/or after the exercise. A unit dosage form contains at least one of oleuropein or metabolite thereof in an amount effective for administration of the unit dosage form before, during and/or after exercise to thereby prevent or treat muscle fatigue from the exercise and/or for resistance to muscle fatigue from exercise. A method of making a composition for preventing or treating muscle fatigue from exercise and/or for resistance to muscle fatigue from exercise, the method including adding an effective amount of at least one of oleuropein or metabolite thereof to at least one ingredient selected from the group consisting of protein, carbohydrate, and fat. The exercise is preferably one or more of 1) resistance exercise, 2) anaerobic or repeated sprint-type exercise, or 3) endurance exercise.

### PRIORITY CLAIM

This application is a continuation-in-part of U.S. Non-Provisional application Ser. No. 17/595,166 filed Nov. 10, 2021, which is a National Stage of International App. No. PCT/EP2020/063329 filed May 13, 2020, which claims priority to U.S. Provisional App. No. 62/847,076 filed May 13, 2019, the entire contents of which are incorporated herein by reference.

### BACKGROUND

The present disclosure generally relates to compositions and methods that use at least one of oleuropein or a metabolite thereof to treat or prevent muscle fatigue from exercise and/or for resistance to muscle fatigue from exercise, for example from one or more of 1) resistance exercise, 2) anaerobic or repeated sprint-type exercise, or 3) endurance exercise.

“Muscle fatigue” means a reduced contractile force in one or more muscles due to a shortage of substrates within the muscle fiber and/or an accumulation of metabolites within the muscle fiber which interfere either with the release of calcium or with the ability of calcium to stimulate muscle contraction. Age-related decrease in muscle mass is due to inter-related factors —lifestyle, structural changes of the muscle, and metabolic changes—and is responsible for almost all loss of strength and power in older adults, with an increase in muscle fatigue.

### SUMMARY

The experimental data disclosed later herein shows the beneficial effect of oleuropein in the context of muscle fatigue from exercise. Accordingly, in some embodiments, the present disclosure provides a method of preventing (e.g., reducing incidence, frequency, and/or severity) or treating muscle fatigue from exercise. Additionally or alternatively, the method provides resistance to muscle fatigue from exercise. The method comprises orally administering at least one of oleuropein or metabolite thereof (e.g., an effective amount) to an individual before, during and/or after exercise by the individual.

Preferably the exercise is at least one of 1) resistance exercise, 2) anaerobic or repeated sprint-type exercise, or 3) endurance exercise. Preferably at least a portion of the muscle cells are part of a skeletal muscle selected from the group consisting of gastrocnemius, tibialis, soleus, extensor digitorum longus (EDL), biceps femoris, semitendinosus, semimembranosus, gluteus maximus, and combinations thereof.

In an embodiment, the at least one of oleuropein or metabolite thereof is administered in at least one dose during at least one time period selected from the group consisting of (i) a pre-exercise time between one hour prior to initiation of the exercise and one second prior to the initiation of the exercise, such as between thirty minutes prior to the initiation of the exercise and one minute prior to the initiation of the exercise, (ii) an exercise time between the initiation of the exercise to conclusion of the exercise, and (i) a post-exercise time between one second after the conclusion of the exercise and one hour after the conclusion of the exercise, such as between one minute after the conclusion of the exercise and thirty minutes after the conclusion of the exercise.

In an embodiment, the metabolite of oleuropein is selected from the group consisting of oleuropein aglycone, hydroxytyrosol, homovanillyl alcohol, isohomovanillyl alcohol, glucuronidated forms thereof, sulfated forms thereof, derivatives thereof, and mixtures thereof.

In an embodiment, the effective amount of at least one of oleuropein or metabolite thereof is administered in a composition selected from the group consisting of food compositions, beverages, dietary supplements, nutritional compositions, nutraceuticals, powdered nutritional products to be reconstituted in water or milk before consumption, food additives, medicaments, drinks, petfood, and combinations thereof.

In an embodiment, the at least one of oleuropein or metabolite thereof is administered in a composition further comprising calcium.

In an embodiment, the effective amount of at least one of oleuropein or metabolite thereof is administered in a food product further comprising a component selected from the group consisting of protein, carbohydrate, fat and mixtures thereof.

In another embodiment, the present disclosure provides a unit dosage form comprising at least one of oleuropein or metabolite thereof in an amount effective for preventing (e.g., reducing incidence, frequency, and/or severity) or treating muscle fatigue from exercise and/or for resistance to muscle fatigue from exercise.

In an embodiment, the unit dosage form consists essentially of the at least one of oleuropein or metabolite thereof.

In an embodiment, the unit dosage form consists of an excipient and the at least one of oleuropein or metabolite thereof.

In an embodiment, the unit dosage form further comprises calcium. The unit dosage form can consist essentially of the calcium and the at least one of oleuropein or metabolite thereof. The unit dosage form can consist of an excipient, the calcium, and the at least one of oleuropein or metabolite thereof.

In another embodiment, the present disclosure provides a method of making a composition for preventing (e.g., reducing incidence, frequency, and/or severity) or treating muscle fatigue from exercise and/or for resistance to muscle fatigue from exercise. The method comprises adding an effective amount of at least one of oleuropein or metabolite thereof to at least one ingredient selected from the group consisting of protein, carbohydrate, and fat. In an embodiment, the method further comprises adding calcium to the at least one ingredient.

In an embodiment, the method further comprises adding to the at least one ingredient a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

Additional features and advantages are described herein and will be apparent from the following Figures and Detailed Description.

### BRIEF DESCRIPTION OF DRAWINGS

FIG. 1 shows the chemical structure of oleuropein.

FIG. 2 shows the proposed metabolism pathway of oleuropein by mammalian and microbial enzymes, based on the findings reported in the literature.

FIG. 3A shows the chemical structure of homovanillyl alcohol; and FIG. 3B shows its isomer (3-hydroxy-4-methoxyphenethanol or 3-hydroxy-4-methoxyphenethyl alcohol).

FIG. 4 is a graph showing that oleuropein increases mitochondrial calcium elevation in Hela cells, during stimulation. Statistical evaluation of the oleuropein (10 μM, black) effect on the integrated mitochondrial calcium rise, evoked by 100 μM histamine. Graph shows the average of 3 independent experiments. Results are expressed as mean +/−SEM. * indicates statistical significant difference vs. control cells (white) at P<0.05 (Student's t-test).

FIG. 5 is a graph showing that oleuropein enhances mitochondrial calcium in caffeine-stimulated myotubes, differentiated from human skeletal muscle myoblasts (HSMM). Statistical evaluation of the oleuropein effect (10 black) on the integrated mitochondrial calcium rise, evoked by 5 mM caffeine. Graph shows the average of 6 independent experiments. Results are expressed as mean +/−SEM. * indicates statistical significant difference vs. control cells (white) at P<0.05 (Student's t-test).

FIG. 6 is a graph showing that metabolites of oleuropein boost mitochondrial calcium in caffeine-stimulated HSMM myotubes. Statistical evaluation of the effect of oleuropein and its metabolites, at 10 μM concentration, on the integrated mitochondrial calcium rise, evoked by 5 mM caffeine. Graph shows the average of 6 independent experiments. Right, selected metabolites. Results are expressed as mean +/−SEM. * indicates statistical significant difference vs. control cells (white) at P<0.05 (one-way ANOVA test).

FIG. 7 is a graph showing that Ca²⁺ supplementation enhances mitochondrial Ca²⁺ elevation in a dose/response manner in C2C12-derived myotubes. Statistical evaluation of the effect of extracellular calcium abundance on the integrated mitochondrial calcium rise, evoked by 5 mM caffeine. Right, calcium concentration in the medium (in mM). Graph shows the average of 12 measurements from 3 independent experiments. Results are expressed as mean +/−SEM. * indicates statistical significant difference vs. 0.5 mM calcium concentration in the medium (white) at P<0.05 (one-way ANOVA test).

FIG. 8 is a graph showing that oleuropein rescues mitochondrial activation in calcium deficiency condition, in C2C12-derived myotubes. Statistical evaluation of the effect of 50 μM oleuropein on the integrated mitochondrial calcium rise, evoked by 5 mM caffeine. Right, calcium concentration in the medium (in mM). Graph shows the average of 12 measurements from 3 independent experiments. Results are expressed as mean +/−SEM. * indicates statistical significant difference vs. 0.5 mM calcium concentration in the medium (white) at P<0.05 (one-way ANOVA test).

FIG. 9 is a graph showing that Oleuropein and hydroxytyrosol boost the ATP-synthase-dependent component of the respiration, during stimulation in myotubes, differentiated from human skeletal muscle (HSM) myoblasts. Statistical evaluation of the effect of 10 μM hydroxytyrosol (gray bar) or 10 μM oleuropein (black bar) on the ATP-synthase-dependent component of the respiration in HSM myotubes, stimulated with 10 μM epibatidine and calculated from the data in the inset. Inset, respiration profile of human skeletal muscle myotubes. The compounds are hydroxytyrosol or oleuropein. Oligomycin was used to determine the ATP-synthase dependent component of the respiration, in epipatidine-stimulated myotubes. Graph shows the average of 8 experiments. Results are expressed as mean +/−SEM. * indicates statistically significant difference vs. control (white bar) at P<0.05 (one-way ANOVA test).

FIG. 10 is a graph showing that Oleuropein increases ATP production in in C2C12-derived myotubes, stimulated with caffeine. Myotubes were incubated with oleuropein for 15 minutes, then they were stimulated with 5 mM caffeine for 10 minutes. Graph shows the average of 8 experiments. Results are expressed as mean +/−SEM. * indicates statistically significant difference vs. control cells (white) at P<0.05 (Student's t-test).

FIG. 11 is a graph showing that Oleuropein increases mitochondrial Calcium uptake in isolated adult mouse myofibers transfected with the mitochondrial calcium sensor 4mtGCaMP6f (ex vivo). Fibers were treated with oleuropein. Three minutes later, cells were stimulated with 60 mM caffeine. Left: representative traces of mitochondrial calcium uptake. Right: mean of mitochondrial calcium peak. Results are expressed as mean +/−SD. * indicates statistically significant difference vs. control myofibers at P<0.05 (Student's t-test), of >20 fibers per condition.

FIG. 12 is a graph showing that hydroxytyrosol increases mitochondrial Calcium uptake in isolated adult mouse myofibers transfected with the mitochondrial calcium sensor 4mtGCaMP6f (ex vivo). Fibers were treated with hydroxytyrosol. Three minutes later, cells were stimulated with 60 mM caffeine. Left: representative traces of mitochondrial calcium uptake. Right: mean of mitochondrial calcium peak. Results are expressed as mean +/−SD. * indicates statistically significant difference vs. control myofibers at P<0.05 (Student's t-test), of >20 fibers per condition.

FIG. 13 is a graph showing that Oleuropein increases mitochondrial respiration in isolated adult mouse myofibers (ex vivo). Fibers treated with Oleuropein for 2 hours, were placed is a XF24 Extracellular Flux Analyzer (Agilent) to measure oxygen consumption rate upon caffeine stimulation. Oligomycin, FCCP and antimycin/rotenone were added consecutively to calculate basal, maximal, ATP-linked and non-mitochondrial respiration. Results are expressed as mean +/−SD. * indicates statistically significant difference vs. control myofibers at P<0.05 (Student's t-test) of 7 wells per condition.

FIG. 14 is a graph showing that Hydroxytyrosol increases mitochondrial respiration in isolated adult mouse myofibers (ex vivo). Fibers treated with Hydroxytyrosol for 2 hours were placed is a XF24 Extracellular Flux Analyzer (Agilent) to measure oxygen consumption rate upon caffeine stimulation. Oligomycin, FCCP and antimycin/rotenone were added consecutively to calculate basal, maximal, ATP-linked and non-mitochondrial respiration. Results are expressed as mean +/−SD. * indicates statistically significant difference vs. control myofibers at P<0.05 (Student's t-test) of 7 wells per condition.

FIG. 15 is a graph showing that Oleuropein increases resistance to fatigue in EDL (Extensor digitorum longus) muscle (ex vivo) from young adult mice. Muscles incubated in Oleuropein show a significantly slower force reduction during fatigue than muscles in which DMSO was added. The third, fourth and fifth tetanic stimulation is significantly higher in Oleuropein compared to control, suggesting a higher resistance to fatigue. The P-value is shown at each tetanic contraction. Results are expressed as mean +/−SD. * indicates statistically significant difference vs. control muscles at P<0.05 (Student's t-test). Each experiment was repeated in 10 muscles for both experimental groups.

FIG. 16 is a graph showing that Bonolive® (BioActor BV, NL), an olive leaf extract standardised for its oleuropein content (≥40% oleuropein and <1% hydroxytyrosol (OHT)), promotes mitochondrial activation by dephosphorylation of Pyruvate dehydrogenase (PDH), in old rats treated for 3 months. The phospho-PDH and PDH levels were analyzed in gastrocnemius muscle in control and 3-months treated animals (inset). The activation of mitochondrial PDH was measured as the ratio between the total PDH and the phospho-PDH level. Graph shows the average of the muscle of 5 animals. Results are expressed as mean +/−SEM. * indicates statistically significant difference vs. control (white bar) at P<0.05 (Student's t-test).

FIG. 17 is a graph showing that chronic in vivo supplementation of oleuropein (standardized at 40%, from olive leaf extract) increases resistance to fatigue in gastrocnemius muscle, from adult mice. Oleuropein show a significantly slower force reduction during fatigue protocol than muscles in which DMSO was added, suggesting a higher resistance to fatigue. Results are expressed as mean +/−SEM. * indicates statistically significant difference vs. control muscles at P<0.05 (Student's t-test). Each experiment was repeated in 10 muscles for both experimental groups.

FIG. 18 is a graph showing that chronic in vivo supplementation of oleuropein (standardized at 40% from olive leaf extract) increases running capacity in young adult mice. Mice were subjected to a single bout of run on an uphill treadmill. Running capacity was measured as the time spent on treadmill, until mice were exhausted. Graph shows the average value of 5 animals per group. Results are expressed as mean +/−SEM. * indicates statistically significant difference vs. control muscles at P<0.05 (Student's t-test).

FIG. 19 is a graph showing that chronic in vivo supplementation of oleuropein (standardized at 40% from olive leaf extract) increases running capacity in aged mice. Mice were subjected to a single bout of run on an uphill treadmill. Running capacity was measured as the time spent on treadmill, until mice were exhausted. Graph shows the average value of 4 animals per group. Results are expressed as mean +/−SEM. * indicates statistically significant difference vs. control muscles at P<0.05 (Student's t-test).

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a metabolite” or “the metabolite” includes one metabolite but also two or more metabolites.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

As used herein, a “composition consisting essentially of at least one of oleuropein or metabolite thereof” and a “composition consisting essentially of calcium and at least one of oleuropein or metabolite thereof” do not include any additional compound that affects mitochondrial calcium import other than the at least one of oleuropein or metabolite thereof and the optional calcium. In a particular non-limiting embodiment, the composition consists of an excipient, the at least one of oleuropein or metabolite thereof, and optionally calcium.

The term “and/or” used in the context of “X and/or Y” should be interpreted as “X,” or “Y,” or “X and Y.” Similarly, “at least one of X or Y” should be interpreted as “X,” or “Y,” or “both X and Y.” For example, “at least one of oleuropein or metabolite thereof” means “oleuropein,” or “a metabolite of oleuropein,” or “both oleuropein and a metabolite thereof.”

Where used herein, the terms “example” and “such as,” particularly when followed by a listing of terms, are merely exemplary and illustrative and should not be deemed to be exclusive or comprehensive. As used herein, “associated with” and “linked with” mean occurring concurrently, preferably means caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development or progression of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

Both human and veterinary treatments are within the scope of the present disclosure. Preferably the at least one of oleuropein or metabolite thereof is administered in a serving or unit dosage form that provides a therapeutically effective or prophylactically effective amount.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

The relative terms “improved,” “increased,” “enhanced” and the like refer to the effects of the composition disclosed herein, namely a composition comprising an effective amount of at least one of oleuropein or metabolite thereof, relative to administration over the same time period of a composition lacking oleuropein and lacking an oleuropein metabolite but otherwise identical.

As used herein, “administering” includes another individual providing a referenced composition to an individual so that the individual can consume the composition and also includes merely the act of the individual themselves consuming a referenced composition.

“Animal” includes, but is not limited to, mammals, which includes but is not limited to rodents; aquatic mammals; domestic animals such as dogs, cats and other pets; farm animals such as sheep, pigs, cows and horses; and humans. Where “animal,” “mammal” or a plural thereof is used, these terms also apply to any animal that is capable of the effect exhibited or intended to be exhibited by the context of the passage, e.g., an animal benefitting from improved mitochondrial calcium import. While the term “individual” or “subject” is often used herein to refer to a human, the present disclosure is not so limited. Accordingly, the term “individual” or “subject” refers to any animal, mammal or human that can benefit from the methods and compositions disclosed herein.

The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. The term “companion animal” means a dog or a cat.

The term “elderly” in the context of a human means an age from birth of at least 60 years, preferably above 63 years, more preferably above 65 years, and most preferably above 70 years. In the context of non-human animals, “elderly” means a non-human subject that has reached 60% of its likely lifespan, in some embodiments at least 70%, at least 80% or at least 90% of its likely lifespan. A determination of lifespan may be based on actuarial tables, calculations, or estimates, and may consider past, present, and future influences or factors that are known to positively or negatively affect lifespan. Consideration of species, gender, size, genetic factors, environmental factors and stressors, present and past health status, past and present nutritional status, and stressors may be taken into consideration when determining lifespan. The term “older adult” in the context of a human means an age from birth of at least 45 years, preferably above 50 years, more preferably above 55 years, and includes elderly individuals.

The terms “serving” or “unit dosage form,” as used herein, are interchangeable and refer to physically discrete units suitable as unitary dosages for human and animal subjects, each unit containing a predetermined quantity of the composition comprising at least one of oleuropein or metabolite thereof, as disclosed herein, in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of liquid housed within a container such as a bottle.

An “oral nutrition supplement” or “ONS” is a composition comprising at least one macronutrient and/or at least one micronutrient, for example in a form of sterile liquids, semi-solids or powders, and intended to supplement other nutritional intake such as that from food. Non-limiting examples of commercially available ONS products include MERITENE®, BOOST®, NUTREN® and SUSTAGEN®. In some embodiments, an ONS can be a beverage in liquid form that can be consumed without further addition of liquid, for example an amount of the liquid that is one serving of the composition.

As used herein, “incomplete nutrition” refers to preferably nutritional products that do not contain sufficient levels of macronutrients (protein, fats and carbohydrates) or micronutrients to be sufficient to be a sole source of nutrition for the animal to which the nutritional product is being administered. The term “complete nutrition” refers to a product which is capable of being the sole source of nutrition for the subject. An individual can receive 100% of their nutritional requirements from a complete nutrition composition.

A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

Preferred embodiments of “exercise” as used herein include at least one of 1) resistance exercise, 2) anaerobic or repeated sprint-type exercise, or 3) endurance exercise.

Resistance exercise is when a subject undertakes explosive movements of weight, with long periods of rest, and is primarily driven by the phosphocreatine and glycolytic energy systems. Resistance exercise can produce energy quickly, but the subject fatigues quickly. The primary adaptations include increases in muscle mass (hypertrophy) by increased muscle cross-section area through repeated weight lifting training. Hakkinen K. 1989. Neuromuscular and hormonal adaptations during strength and power training. J. Sports Med. Phys. Fitness. 29:9-26; and Hakkinen K. et. al. 1987. Relationships between training volume, physical performance capacity, and serum hormone concentrations during prolonged training in elite weight lifters. Int. J. Sports Med. 8 Suppl 1:61-65.

Repeated sprint-type training is anaerobic, involves high-intensity exercise with limited recovery periods, and involves nearly purely carbohydrate metabolism with a large breakdown in muscle glycogen (glycolytic energy production). During these situations of anaerobic energy production, such as high intensity speed training or sports involving repeated sprints, the increased load on the muscles is accomplished by an increased firing of Type IIa fibers. Finally, at very high workloads, type IIb glycolytic muscle fibers become activated to maintain the high demand of energy provision via anaerobic energy provision. However, during these situations, the high rate of anaerobic energy production exceeds the rate at which it can be oxidized aerobically within the mitochondria, and this leads to the extreme levels of lactate production found in these types of training situations. Spriet L L, Howlett R A, and Heigenhauser G J. 2000. An enzymatic approach to lactate production in human skeletal muscle during exercise. Med. Sci. Sports Exerc. 32: 756-763.

Endurance training is characterized by individuals performing low-intensity training over prolonged periods (e.g., >15 minutes). The energy system represented for endurance training includes the aerobic system, which primarily uses aerobic metabolism of fats and carbohydrates to produce the required energy within the mitochondria when ample oxygen is present. The primary adaptations include increased muscle glycogen stores and glycogen sparing at sub-maximal workloads via increased fat oxidation, enhanced lactate kinetics and morphological alterations, including greater type I fiber per muscle area, and increased capillary and mitochondrial density. Holloszy J 0, and Coyle E F. 1984. Adaptations of skeletal muscle to endurance exercise and their metabolic consequences. J. Appl. Physiol. 56: 831-838; and Holloszy J O, Rennie M J, Hickson R C, Conlee R K, and Hagberg J M. 1977. Physiological consequences of the biochemical adaptations to endurance exercise. Ann. N.Y. Acad. Sci. 301: 440-450.

### EMBODIMENTS

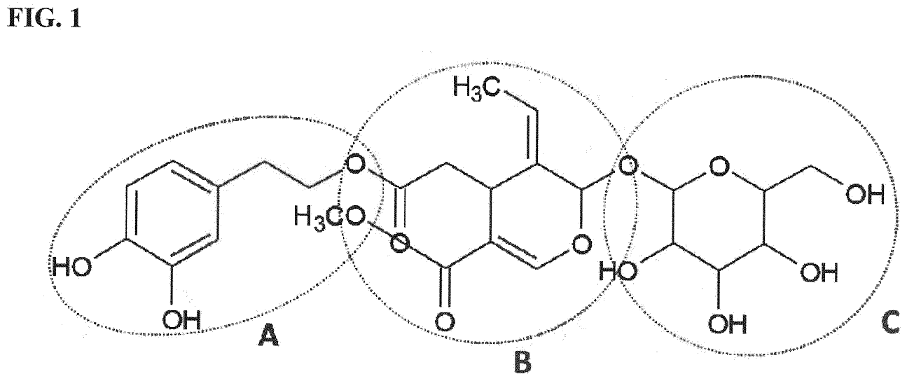

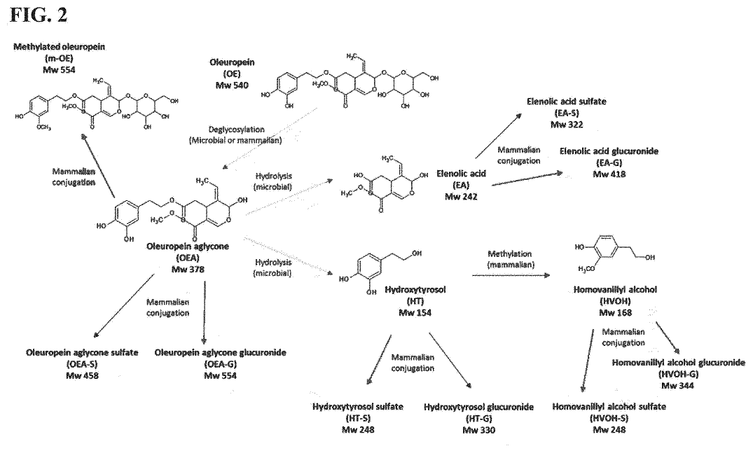

Oleuropein is a polyphenol found in the fruit, the roots, the trunk and more particularly in the leaves of plants belonging to the Oleaceae family, and especially Olea europaea. FIG. 1 shows the chemical structure of oleuropein. Oleuropein is a heterosidic ester of 3, 4-dihydroxyphenylethanol (also known as hydroxytyrosol, labeled as “A” in FIG. 1) and elenolic acid (labeled as “B” in FIG. 1) containing a molecule of glucose (labeled as “C” in FIG. 1). FIG. 2 shows a proposed metabolism pathway of oleuropein by mammalian and microbial enzymes, based on the findings reported in the literature.

An aspect of the present disclosure is a method of preventing or treating muscle fatigue from exercise and/or for resistance to muscle fatigue from exercise. The method comprises orally administering an effective amount of at least one of oleuropein or metabolite thereof to an individual before, during and/or after the exercise. Preferably, the metabolite of oleuropein is selected from the group consisting of oleuropein aglycone, hydroxytyrosol, homovanillyl alcohol, isohomovanillyl alcohol, glucuronidated forms thereof, sulfated forms thereof, derivatives thereof, and mixtures thereof.

In some embodiments, the effective amount of at least one of oleuropein or metabolite thereof is administered in a composition selected from the group consisting of food compositions, dietary supplements, nutritional compositions, nutraceuticals, beverages, powdered nutritional products to be reconstituted in water or milk before consumption, food additives, medicaments, drinks, petfood, and combinations thereof.

The effective amount of at least one of oleuropein or metabolite thereof is preferably administered in a composition further comprising calcium.

In some embodiments, the effective amount of at least one of oleuropein or metabolite thereof is administered in a food product further comprising a component selected from the group consisting of protein, carbohydrate, fat and mixtures thereof.

The exercise is preferably one or more of 1) resistance exercise, 2) anaerobic or repeated sprint-type exercise, or 3) endurance exercise.

Another aspect of the present disclosure is a unit dosage form comprising at least one of oleuropein or metabolite thereof in an amount effective for administration of the unit dosage form before, during and/or after exercise to thereby prevent or treat muscle fatigue from the exercise and/or for resistance to muscle fatigue from exercise. In some embodiments, the unit dosage form (i) consists essentially of the at least one of oleuropein or metabolite thereof, (ii) consists of an excipient and the at least one of oleuropein or metabolite thereof, (iii) consists essentially of calcium and the at least one of oleuropein or metabolite thereof, or (iv) consists of an excipient, calcium, and the at least one of oleuropein or metabolite thereof. Preferably the exercise is one or more of 1) resistance exercise, 2) anaerobic or repeated sprint-type exercise, or 3) endurance exercise.

Yet another aspect of the present disclosure is a method of making a composition for preventing or treating muscle fatigue from exercise and/or for resistance to muscle fatigue from exercise. The method comprises adding an effective amount of at least one of oleuropein or metabolite thereof to at least one ingredient selected from the group consisting of protein, carbohydrate, and fat. In some embodiments, the method further comprises adding to the at least one ingredient a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof. Preferably the method further comprises adding calcium to the at least one ingredient. Preferably the exercise is one or more of 1) resistance exercise, 2) anaerobic or repeated sprint-type exercise, or 3) endurance exercise.

Another aspect of the present disclosure is a method of achieving at least one result selected from the group consisting of (i) improvement in a physiological state linked to metabolic fatigue in one or more cells, (ii) increased mitochondrial energy and mitochondrial calcium uptake in one or more cells, and (iii) treatment or prevention of a calcium deficiency/depletion disorder (e.g., reduction in incidence and/or severity). The method comprises orally administering an effective amount of at least one of oleuropein or metabolite thereof to an individual.

Another aspect of the present disclosure is a method of treating in an individual in need thereof or preventing in an individual at risk thereof (e.g., reducing incidence and/or severity) at least one condition selected from the group consisting of (i) a physiological state linked to metabolic fatigue in one or more cells and (ii) a calcium deficiency/depletion disorder. The method comprises orally administering an effective amount of at least one of oleuropein or metabolite thereof to the individual in need thereof or at risk thereof.

Yet another aspect of the present disclosure is a method of treating or preventing (e.g., reducing incidence and/or severity) a mitochondria-related disease or a condition associated with altered mitochondrial function in an individual in need thereof or at risk thereof. The method comprises orally administering to an individual an effective amount of at least one of oleuropein or metabolite thereof. The mitochondria-related disease or condition can be selected from the group consisting of stress, physiological ageing, obesity, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, chronic kidney disease, kidney failure, trauma, infection, cancer, hearing loss, macular degeneration, myopathies and dystrophies, and combinations thereof.

In another embodiment, metabolic fatigue comprises lack of energy, in particular physical energy, lack of vitality or weakness.

In some embodiments, the methods comprise identifying the individual as having the condition or being at risk of the condition before the administration.

The effective amount of the at least one of oleuropein or metabolite thereof varies with the particular composition, the age and condition of the recipient, and the particular disorder or disease being treated. Nevertheless, in a general embodiment, 0.001 mg to 1.0 g of the at least one of oleuropein or metabolite thereof can be administered to the individual per day, preferably from 0.01 mg to 0.9 g of the at least one of oleuropein or metabolite thereof per day, more preferably from 0.1 mg to 750 mg of the at least one of oleuropein or metabolite thereof per day, more preferably from 0.5 mg to 500 mg of the at least one of oleuropein or metabolite thereof per day, and most preferably from 1.0 mg to 200 mg of the at least one of oleuropein or metabolite thereof per day.

In some embodiments, the oleuropein is administered in a composition further comprising calcium. At least a portion of the calcium can be one or more calcium salts, such as calcium acetate, calcium carbonate, calcium chloride, calcium citrate, calcium glubionate, calcium gluconate, calcium lactate or mixtures thereof. In a general embodiment, 0.1 g to 1.0 g of the calcium is administered to the individual per day, preferably from 125 mg to 950 g of the calcium per day, more preferably from 150 mg to 900 mg of the calcium per day, more preferably from 175 mg to 850 mg of the calcium per day, and most preferably from 200 mg-800 mg of the calcium per day.

In an embodiment, at least a portion of the oleuropein is obtained by extraction, e.g., by extraction from a plant such as a plant belonging to the Oleaceae family, preferably one or more of the stems, the leaves, the fruits or the stones of a plant belonging to the Oleaceae family such as Olea europaea (olive tree), a plant of genus Ligustrum, a plant of genus Syringa, a plant of genus Fraximus, a plant of genus Jasminum and a plant of genus Osmanthus. Additionally or alternatively, at least a portion of the oleuropein and/or metabolites can be obtained by chemical synthesis.

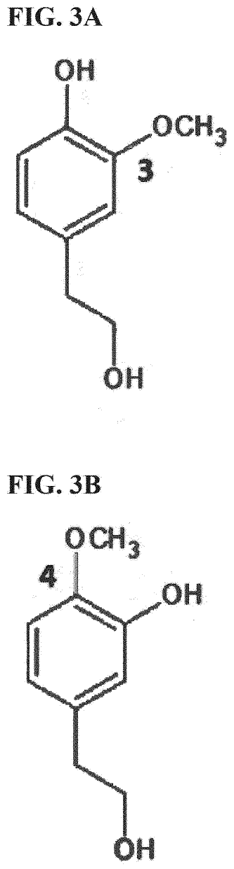

Non-limiting examples of suitable metabolites of oleuropein include oleuropein aglycone, hydroxytyrosol, homovanillyl alcohol, isohomovanillyl alcohol, glucuronidated forms thereof, sulfated forms thereof, derivatives thereof, and mixtures thereof. FIG. 3A shows the chemical structure of homovanillyl alcohol; and FIG. 3B shows its isomer (3-hydroxy-4-methoxyphenethanol or 3-hydroxy-4-methoxyphenethyl alcohol).

In some embodiments, the at least one of oleuropein or metabolite thereof is the only polyphenol in the composition and/or the only polyphenol administered to the individual.

In some embodiments, the at least one of oleuropein or metabolite thereof and the optional calcium can be administered to an elderly subject. In some embodiments, the individual is healthy. In some embodiments, the individual has metabolic fatigue, but optionally is otherwise healthy. In some embodiments, the individual can be a pet.

In an embodiment, at least a portion of the one or more cells are part of at least one body part selected from the group consisting of liver, kidney, brain and skeletal muscle.

The at least one of oleuropein or metabolite thereof and the optional calcium can be administered in any composition that is suitable for human and/or animal consumption. In a preferred embodiment, the at least one of oleuropein or metabolite thereof and the optional calcium is administered to the individual orally or enterally (e.g. tube feeding). For example, the at least one of oleuropein or metabolite thereof and the optional calcium can be administered to the individual in a beverage, a food product, a capsule, a tablet, a powder or a suspension.

Non-limiting examples of suitable compositions for the include food compositions, dietary supplements, dietary supplements (e.g., liquid ONS), complete nutritional compositions, beverages, pharmaceuticals, nutraceuticals, powdered nutritional products to be reconstituted in water or milk before consumption, food additives, medicaments, drinks, petfood, and combinations thereof.

Food products according to the present invention may include dairy products, such as fermented milk products, e.g., yoghurts, buttermilk, etc; ice creams; concentrated milk; milk; dairy creams; flavoured milk drinks; whey based drinks; toppings; coffee creamers; chocolate; cheese based products; soups; sauces; purees; dressings; puddings; custards; baby foods; nutritional formulas, such as those for complete nutrition, for example for infants, children, teenagers, adults, the elderly or the critically ill; cereals and cereal bars, for example.

Drinks may include for example milk- or yoghurt based drinks, fermented milk, protein drinks, coffee, tea, energy drinks, soy drinks, fruit and/or vegetable drinks, fruit and/or vegetable juices.

The at least one of oleuropein or metabolite thereof and the optional calcium can be administered in a food product further comprising a component selected from the group consisting of protein, carbohydrate, fat and mixtures thereof.

In some instances where oral or enteral administration is not possible or not advised, the composition may be administered parenterally.

In another embodiment, the present disclosure provides a method of treating or preventing (e.g., reducing incidence and/or severity) a mitochondria-related disease or a condition associated with altered mitochondrial function in an individual in need thereof or at risk thereof. The method comprises orally administering an effective amount of at least one of oleuropein or metabolite thereof to the individual in need thereof or at risk thereof.

In an embodiment, the at least one of oleuropein or metabolite thereof and the optional calcium is administered to the individual for a time period of at least one month; preferably at least two months, more preferably at least three, four, five or six months; most preferably for at least one year. During the time period, the at least one of oleuropein or metabolite thereof and the optional calcium can be administered to the individual at least one day per week; preferably at least two days per week, more preferably at least three, four, five or six days per week; most preferably seven days per week. The at least one of oleuropein or metabolite thereof and the optional calcium can be administered in a single dose per day or in multiple separate doses per day.

The above examples of administration do not require continuous daily administration with no interruptions. Instead, there may be some short breaks in the administration, such as a break of two to four days during the period of administration. The ideal duration of the administration of the composition can be determined by those of skill in the art.

In an embodiment, the at least one of oleuropein or metabolite thereof can be administered with calcium in the same composition, for example a unit dosage form containing both the calcium and the at least one of oleuropein or metabolite thereof.

In an alternative embodiment, the at least one of oleuropein or metabolite thereof can be administered sequentially with calcium in separate compositions. The term “sequentially” means that the calcium and the at least one of oleuropein or metabolite thereof are administered in a successive manner such that the at least one of oleuropein or metabolite thereof is administered at a first time without the calcium, and the calcium is administered at a second time (before or subsequent to the first time) without the at least one of oleuropein or metabolite thereof. The time between sequential administrations may be, for example, one or several seconds, minutes or hours in the same day; one or several days or weeks in the same month; or one or several months in the same year.

Another aspect of the present disclosure is a method of making a composition for achieving an effect selected from the group consisting of (i) improvement in a physiological state linked to metabolic fatigue in one or more cells, (ii) increased mitochondrial energy and mitochondrial calcium uptake in one or more cells, and (iii) treatment or prevention of a calcium deficiency/depletion disorder (e.g., reduction in incidence and/or severity).

The method comprises adding at least one of oleuropein or metabolite thereof to an ingredient selected from the group consisting of a protein, a carbohydrate, a lipid, and combinations thereof. The composition (e.g., food product) can be made prior to administration (e.g., the composition is made, packaged, and then purchased by a consumer who administers the composition to themselves or to another individual) or can be made substantially simultaneous to administration (the composition is made less than 30 minutes before administration, preferably less than 15 minutes before administration, more preferably less than 10 minutes before administration, most preferably less than 5 minutes before administration, by an individual who administers the composition to themselves or to another individual).

The composition can comprise an effective amount of at least one of oleuropein or metabolite thereof. For example, a single serving or dose of the composition can comprise the effective amount, and a package can contain one or more of the servings or doses. Optionally the composition can further comprise calcium.

The composition can comprise a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

In addition to the at least one of oleuropein or metabolite thereof and the optional calcium, the composition can further comprise a protein source from animal or plant origin, for example milk proteins, soy proteins, and/or pea proteins. In a preferred embodiment, the protein source is selected from the group consisting of whey protein; casein protein; pea protein; soy protein; wheat protein; corn protein; rice protein; proteins from legumes, cereals and grains; and combinations thereof. Additionally or alternatively, the protein source may comprise a protein from nuts and/or seeds.

The protein source preferably comprises whey protein. The whey protein may be unhydrolyzed or hydrolyzed whey protein. The whey protein may be any whey protein, for example the whey protein can be selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof. In a preferred embodiment, the whey protein comprises whey protein isolate and/or modified sweet whey.

As noted above, the protein source can be from animal or plant origin, for example milk proteins, soy proteins, and/or pea proteins. In an embodiment, the protein source comprises casein. Casein may be obtained from any mammal but is preferably obtained from cow milk and preferably as micellar casein.

The composition can comprise one or more branched chain amino acids. For example, the composition can comprise leucine, isoleucine and/or valine. The protein source in the composition may comprise leucine in free form and/or leucine bound as peptides and/or proteins such as dairy, animal or vegetable proteins. In an embodiment, the composition comprises the leucine in an amount up to 10 wt % of the dry matter of the composition. Leucine can be present as D- or L-leucine and preferably the L-form. If the composition comprises leucine, the composition can be administered in a daily dose that provides 0.01 to 0.04 g of the leucine per kg body weight, preferably 0.02 to 0.035 g of the leucine per kg body weight. Such doses are particularly applicable to complete nutrition compositions, but one of ordinary skill will readily recognize how to adapt these doses for an oral nutritional supplement (ONS).

One or more other minerals additional to any calcium can be used in the composition. Non-limiting examples of suitable minerals include boron, chromium, copper, iodine, iron, magnesium, manganese, molybdenum, nickel, phosphorus, potassium, selenium, silicon, tin, vanadium, zinc, and combinations thereof.

One or more other vitamins additional to any can be used in the composition. Non-limiting examples of suitable vitamins include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), Vitamin C, Vitamin D, Vitamin E, Vitamin K, folic acid and biotin), and combinations thereof. “Vitamin” includes such compounds obtained naturally from plant and animal foods or synthetically made, pro-vitamins, derivatives thereof, and analogs thereof.

The composition may also contain a carbohydrate and/or a source of fat. Non-limiting examples of suitable fats include canola oil, corn oil and high-oleic acid sunflower oil. Non-limiting examples of suitable carbohydrates include sucrose, lactose, glucose, fructose, corn syrup solids, maltodextrins, and mixtures thereof. Additionally or alternatively, a dietary fiber may be added. Dietary fiber passes through the small intestine undigested by enzymes and functions as a natural bulking agent and laxative. Dietary fiber may be soluble or insoluble and generally a blend of the two types is preferred. Non-limiting examples of suitable dietary fibers include soy, pea, oat, pectin, guar gum, partially hydrolyzed guar gum, gum Arabic, fructo-oligosaccharides, acidic oligosaccharides, galacto-oligosaccharides, sialyl-lactose and oligosaccharides derived from animal milks. A preferred fiber blend is a mixture of inulin with shorter chain fructo-oligosaccharides. In an embodiment, the fiber content is between 2 and 40 g/L of the composition, for example between 4 and 10 g/L.

One or more food grade emulsifiers may be incorporated into the composition, such as diacetyl tartaric acid esters of mono- and di-glycerides, lecithin, and/or mono- and di-glycerides. Suitable salts and stabilizers may be included.

### EXAMPLES

The following non-limiting examples present experimental data supporting the compositions and methods disclosed herein.

### Example 1

To test the effect of Oleuropein, its metabolites and calcium supplementation/deficiency in living cells, the inventors measured mitochondrial calcium elevation in HeLa cells and in myotubes differentiated from both mouse C2C12 cells and human primary adult muscle cells. HeLa cells and C2C12 cells were purchased from ATCC. Human Skeletal Muscle Myoblasts (HSMM) were purchased from Lonza. HSMM were isolated from the upper arm or leg muscle tissue of normal donors and used after the second passage. HeLa cells were seeded in 96-well plates at a density of 50000 cells per well in minimal essential medium (DMEM, Gibco), high glucose, +10% fetal calf serum. C2C12 cells were seeded in 96-well plates at a density of 8000 cells per well in DMEM high glucose (Gibco)+10% fetal calf serum. Myotubes were differentiated from C2C12 cells by growing the cells in DMEM containing 2% horse serum, for 4 days. HSMM were seeded in 96-well plates at a density of 8000 cells per well in DMEM/F-12 (Gibco). Myotubes were differentiated from HSMM by growing the cells in SKM-M medium (ZenBio) containing 2% horse serum, for 4 days.

Mitochondrial calcium measurements were carried out using Hela cells or myotubes infected with the adenovirus (from Sirion biotech) expressing the mitochondrially targeted calcium sensor mitochondrial mutated aequorin (Montero et al., 2004). For aequorin reconstitution, 24 hours after infection, cells or myotubes were incubated for 2 h at room temperature (22±° C.) in standard medium (145 mM NaCl, 5 mM KCl, 1 mM MgCl₂, 1 mM CaCl₂), 10 mM glucose and 10 mM Hepes, pH 7.4) with 1 μM wild-type coelenterazine.

For treatment, compounds were directly added to the cell culture or myotubes cultures 2 hours before measurements. Luminescence was measured at the Cytation 3 cell imaging reader (Biotek) or at the FLIPR Tetra Aequorin (Molecular Devices). Calibration of the luminecsnce data into Calcium concentration was carried out using an algoritm as described previously (Alvarez & Montero, 2002). Custom module analysis based on Excel (Microsoft) and GhaphPad Prism 7.02 (GraphPad) software was used for quantification.

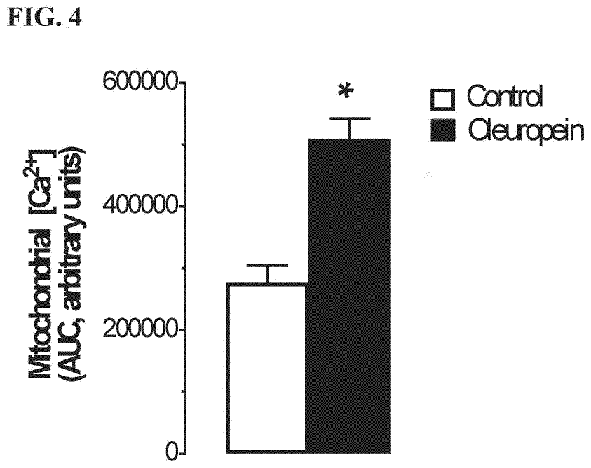

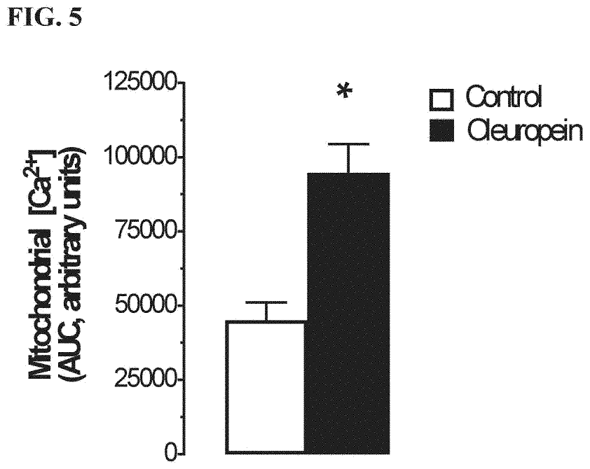

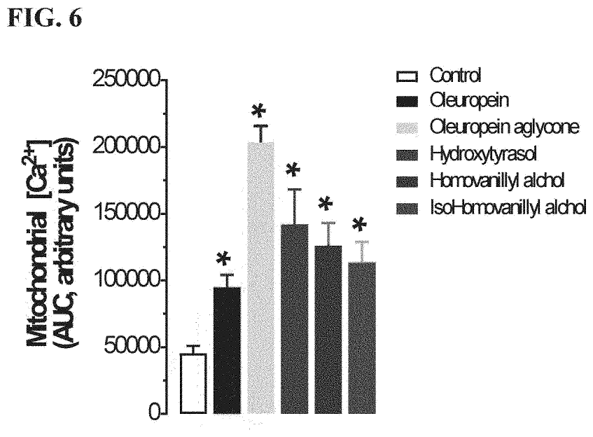

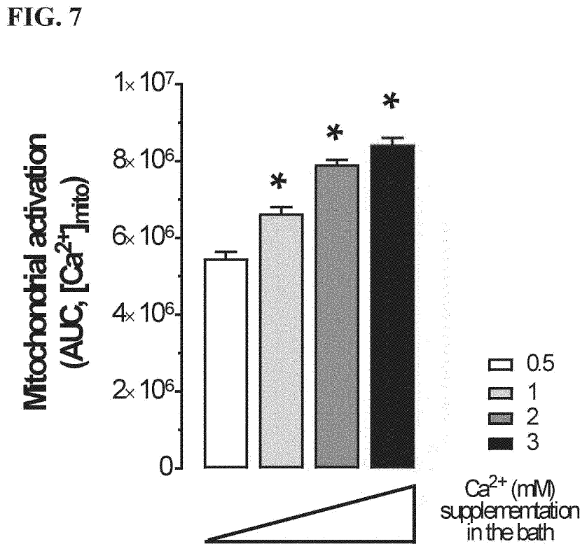

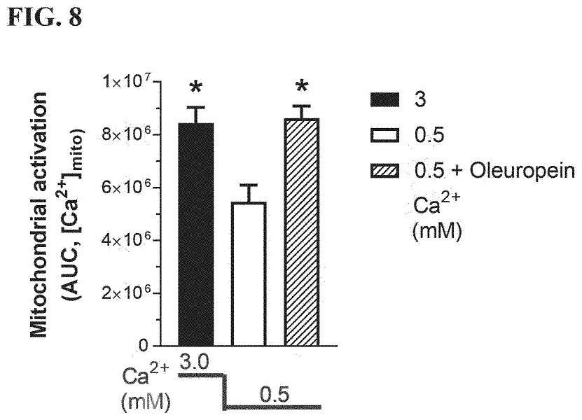

As shown in FIG. 4, oleuropein increases mitochondrial calcium elevation in Hela cells, during stimulation. As shown in FIG. 5, oleuropein activates mitochondrial calcium in caffeine-stimulated human myotubes, differentiated from human skeletal muscle myoblasts (HSMM). As shown in FIG. 6, phenolic metabolites of oleuropein activate mitochondrial calcium in caffeine-stimulated HSMM myotubes. As shown in FIG. 7, Ca²⁺ supplementation activates mitochondrial Ca²⁺ elevation in a dose/response manner in C2C12-derived myotubes. As shown in FIG. 8, oleuropein rescues mitochondrial activation in calcium depletion condition, in C2C12-derived myotubes.

### Example 2

To test the effect of oleuropein and hydroxytyrosol on mitochondrial respiration and to evaluate the effect of these compounds on the ATP-synthase-dependent component of the respiration, the inventors measured oxygen consumption in human skeletal muscle myotubes. For respiration experiments, oxygen consumption was measured in myotubes using a XF96 instrument (Seahorse Biosciences, MA). Human myotubes were seeded into polyornithine-coated Seahorse tissue plates at and after 2 days, the cells were washed twice in Krebs-Ringer bicarbonate Hepes buffer (KRBH), containing (in mM): 140 NaCl, 3.6 KCl, 0.5 NaH₂PO₄, 0.5 MgSO₄, 1.5 CaCl₂), 10 Hepes, 5 NaHCO₃, 10 glucose, pH 7.4. Respiration rates were determined every 6 min at 37° C. ATP synthase-dependent respiration was calculated as the difference in respiration rate before and after the addition of oligomycin. The experiments were performed at 37° C.

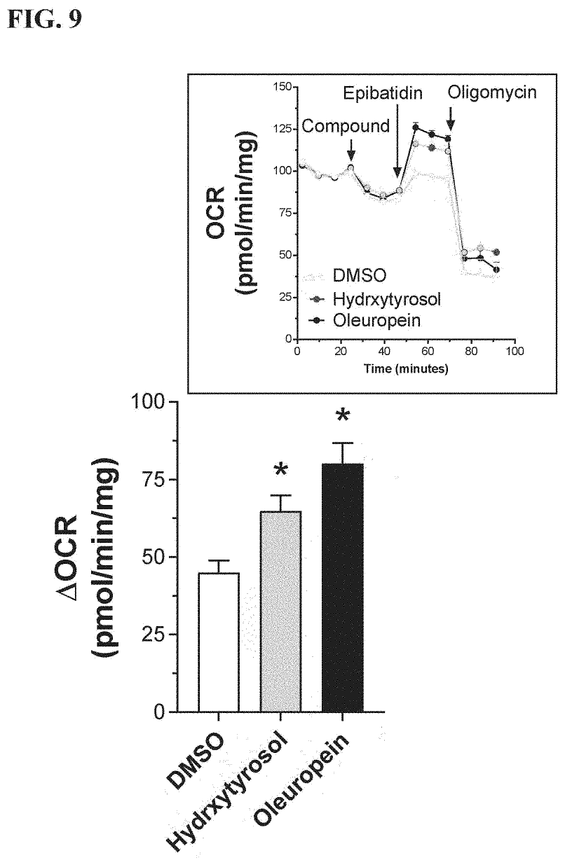

As shown in FIG. 9, oleuropein and hydroxytyrosol boost the ATP-synthase-dependent component of the respiration, during stimulation in human skeletal muscle myotubes.

### Example 3

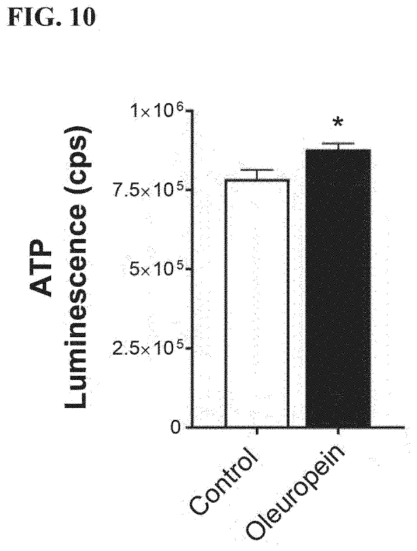

To test the effect of oleuropein on ATP production, the inventors measured ATP in myotubes differentiated from C2C12 cells. ATP was measured with conventional luminescence-based luciferin/luciferase method. Myotubes were incubated in KRBH medium and oleuropein was added for 15 minutes. Then myotubes were stimulated with 5 mM caffeine for additional 10 minutes. Finally, myotubes were incubated with luciferin/luciferase in lysis buffer and bioluminescence signal proportional to the amount of ATP present was measured at the Cytation 3 cell imaging reader (Biotek). As shown in FIG. 10 oleuropein increases ATP production in in C2C12-derived myotubes, stimulated with caffeine.

### Example 4

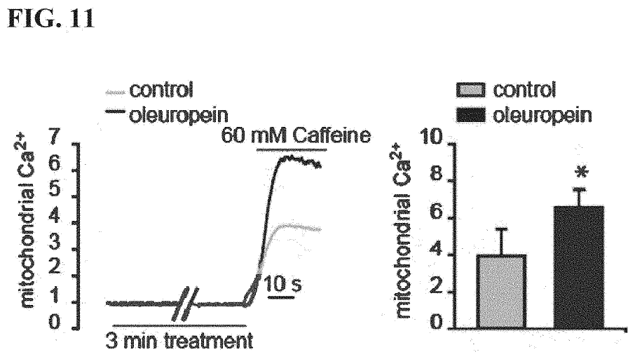

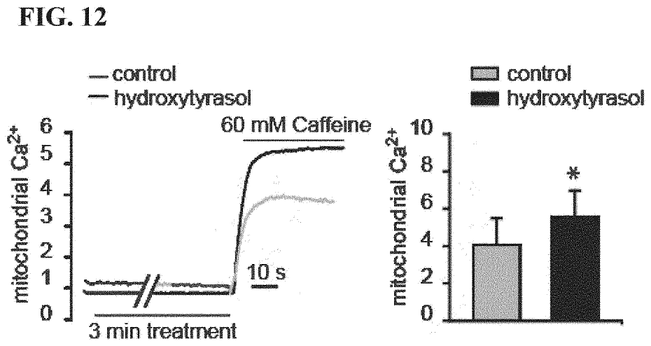

To test the effect of oleuropein and hydroxytyrosol on mitochondrial calcium uptake in isolated adult mouse myofibers, flexor digitorum brevis (FDB) fibers were isolated 7-10 days after in vivo transfection. Muscles were digested in collagenase A (4 mg/ml) (Roche) dissolved in Tyrode's salt solution (pH 7.4) (Sigma-Aldrich) containing 10% fetal bovine serum (Thermo Fisher Scientific). Single fibers were isolated, plated on laminin-coated glass coverslips and cultured in DMEM with HEPES (42430 Thermo Fisher Scientific), supplemented with 10% fetal bovine serum, containing penicillin (100 U/ml), streptomycin (100 μg/ml). Fibers were maintained in culture at 37° C. with 5% CO₂. For mitochondrial Ca²⁺ measurements, FDB muscles were electroporated with a plasmid encoding the mitochondrial calcium sensor 4mtGCaMP6f. After single fibers isolation, real time imaging was performed. During the experiments, myofibers were maintained in Krebs-Ringer modified buffer (135 mM NaCl, 5 mM KCl, 1 mM MgCl₂, 20 mM HEPES, 1 mM MgSO₄, 0.4 mM KH₂PO₄, 1 mM CaCl₂, 5.5 mM glucose, pH 7.4) at room temperature, in presence of 75 μM N-benzyl-P-toluenesulfonamide (BTS, Sigma-Aldrich) to avoid fiber contraction. 60 mM caffeine (Sigma-Aldrich) was added when indicated to elicit calcium release from intracellular stores. Experiments were performed on a Zeiss Axiovert 200 microscope equipped with a 40×/1.3 N.A. PlanFluor objective. Excitation was performed with a DeltaRAM V high-speed monochromator (Photon Technology International) equipped with a 75 W xenon arc lamp. Images were captured with a high-sensitivity Evolve 512 Delta EMCCD (Photometrics). The system is controlled by MetaMorph 7.5 (Molecular Devices) and was assembled by Crisel Instruments. 4mtGCaMP6f sensor was alternatively excited every second at 410 and 475 nm respectively and images were acquired through a dual band emission filter (520/40 and 630/60) (Chroma). Exposure time was set to 50 ms. Acquisition was performed at binning 1 with 200 of EM gain. Image analysis was performed with Fiji distribution of the ImageJ software. Images were background subtracted. As shown in FIG. 11 oleuropein increases mitochondrial calcium uptake in isolated adult mouse myofibers. As shown in FIG. 12 oleuropein increases mitochondrial calcium uptake in isolated adult mouse myofibers.

### Example 5

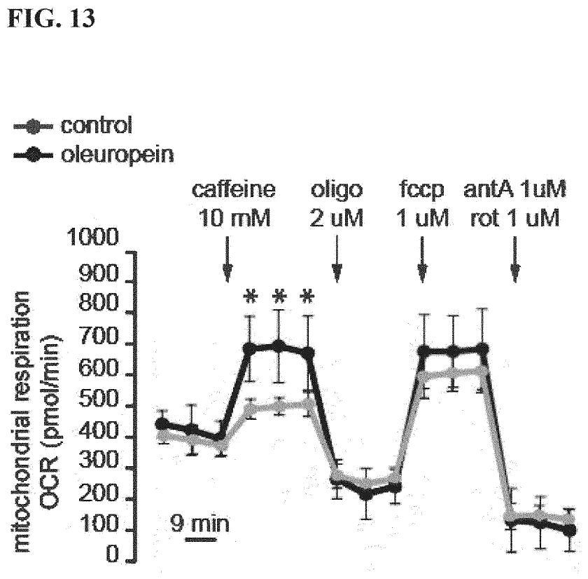

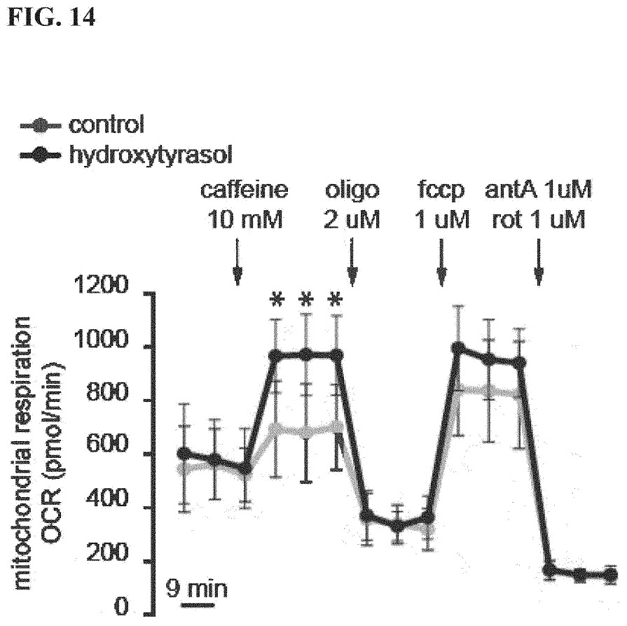

To test the effect of oleuropein and hydroxytyrosol on mitochondrial respiration in isolated adult mouse myofibers, flexor digitorum brevis (FDB) fibers were isolated as follows. Muscles were digested in collagenase A (4 mg/ml) (Roche) dissolved in Tyrode's salt solution (pH 7.4) (Sigma-Aldrich) containing 10% fetal bovine serum (Thermo Fisher Scientific). Single fibers were isolated, plated on laminin-coated XF24 microplate wells and cultured in DMEM (D5030 Sigma-Aldrich), supplemented with 1 mM Na Pyruvate, 5 mM glucose, 33 mM NaCl, 15 mg phenol red, 25 mM HEPES, 1 mM of L-Glu in presence of 75 μM N-benzyl-P-toluenesulfonamide (BTS, Sigma-Aldrich). Fibers were maintained for 2 hours in culture at 37° C. in 5% CO₂. The rate of oxygen consumption was assessed in real-time with the XF24 Extracellular Flux Analyzer (Agilent), which allows to measure oxygen consumption rate (OCR) changes after up to four sequential additions of compounds. A titration with the uncoupler FCCP was performed, in order to utilize the FCCP concentration (0.6 μM) that maximally increases OCR. To measure the effect of oleuropein or hydroxytyrosol on the stimulated respiration, fibers were stimulated with 10 mM caffeine. To evaluate the ATP-synthase-dependent component of the respiration, oligomycin (2 □M) was added. The results were normalized for the fluorescence of Calcein (Sigma-Aldrich). Fibers were loaded with 2 μM Calcein for 30 min. Fluorescence was measured using a Perkin Elmer EnVision plate reader in well scan mode using 480/20 nm filter for excitation and 535/20 nm filter for emission. As shown in FIG. 13, oleuropein increases the stimulated mitochondrial respiration and the ATP-synthase-dependent component of the respiration in isolated adult mouse myofibers. As shown in FIG. 14, hydroxytyrosol increases the stimulated mitochondrial respiration and the ATP-synthase-dependent respiration in isolated adult mouse myofibers.

### Example 6

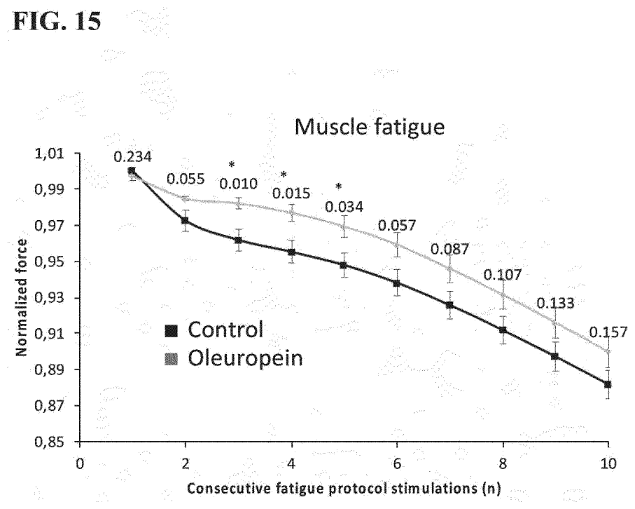

To test the acute effect of oleuropein on muscle fatigue in healthy adult mice, extensor digitorum longus (EDL) muscles were dissected from tendon to tendon under a stereomicroscope and mounted between a force transducer (KG Scientific Instruments, Heidelberg, Germany) in a small chamber in which oxygenated Krebs solution was continuously circulated and temperature maintained at 25° C. The stimulation conditions were optimized, and the length of the muscle was increased until force development during a 90 Hz stimulation was maximal. oleuropein was added to the medium at a final concentration of 10 μM after the measurement of the first force-frequency relationship. Next, the force-frequency was determined every 10 minutes up to one hour after addition. After one hour, fatiguing protocol, which consisted of 120 tetanic contractions (100 Hz) with a duration of 300 ms repeated every second, was applied. Fatigue was determined as the force reduction relative to the initial force. Each experiment was repeated in 10 muscles for both experimental groups. As shown in FIG. 15, muscles incubated in oleuropein show a significantly slower force reduction during fatigue than in control muscles, indicating increased resistance to fatigue.

### Example 7

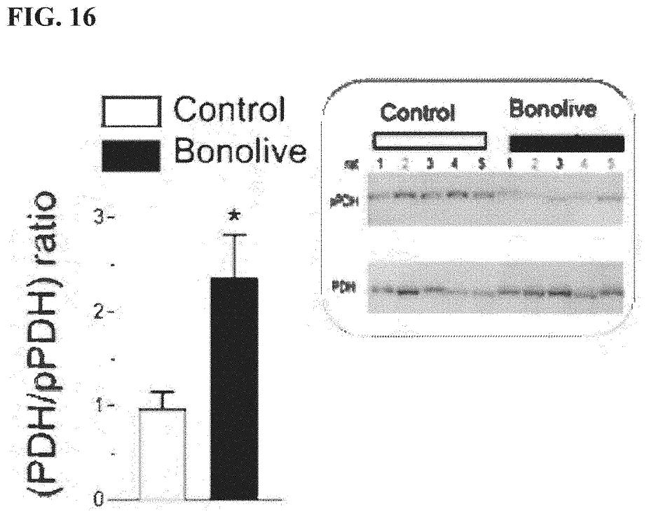

To test the effect an olive leaf extract standardised for its oleuropein content (≥40% oleuropein), on mitochondrial activation by dephosphorylation of the pyruvate dehydrogenase (PDH), 20 months-old rats were supplemented for 3 months with Bonolive® (BioActor BV, NL), then the gastrocnemius muscles were analyzed by western. The content of PDH and phospho-PDH were quantified. To monitor protein levels, frozen muscles were pulverized by means of Qiagen Tissue Lyser and protein extracts were prepared in an appropriate buffer containing: muscle lysis buffer (50 mM Tris pH 7.5, 150 mM NaCl, 5 mM MgCl₂, 1 mM DTT, 10% glycerol, 2% SDS, 1% Triton X-100, Complete EDTA-free protease inhibitor mixture (Roche), 1 mM PMSF, 1 mM NaVO3, 5 mM NaF and 3 mM β-glycerophosphate). 40 μg of total proteins were loaded, according to BCA quantification. Proteins were separated by SDS-PAGE electrophoresis, in commercial 4-12% acrylamide gels (Thermo Fisher Scientific) and transferred onto nitrocellulose membranes (Thermo Fisher Scientific) by wet electrophoretic transfer. Blots were blocked 1 hour at RT with 5% non-fat dry milk (Bio-Rad) in TBS-tween (0.5M Tris, 1.5M NaCl, 0.01% Tween) solution and incubated at 4° C. with primary antibodies. Secondary antibodies were incubated 1 hr at RT. The following antibodies were used: anti-phosphoPDH (1:5000, Abcam), anti-PDH (1:1000, Cell Signaling). Secondary HRP-conjugated antibodies were purchased from Bio-Rad and used at 1:5000 dilution. The activation of mitochondrial PDH was measured as the ratio between the total PDH and the phospho-PDH level. As shown in FIG. 16, Bonolive® (BioActor BV, NL), promotes mitochondrial activation by dephosphorylation of Pyruvate dehydrogenase, in old rats supplemented for 3 months with Bonolive® (BioActor BV, NL).

### Example 8

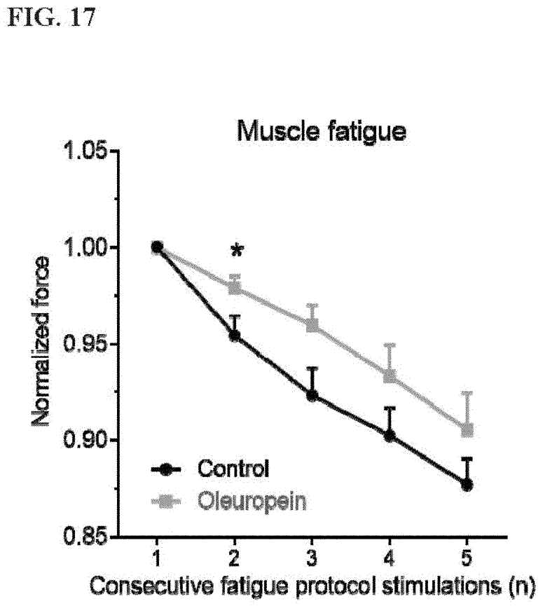

Experiments and conditions on the beneficial effect of oleuropein in vivo, in the context of muscle fatigue. FIGS. 17-19 are different figures, representative of three different conditions (one figure with electrophysiology and two figures on treadmill).

To test the chronic effect of oleuropein on muscle fatigue in healthy adult mice (FIG. 17), 2 months old mice were supplemented daily for 4 weeks with olive leaf extract, standardised for its oleuropein content (20 mg/kg/day oleuropein). The fatigue protocol was performed in gastrocnemius muscle and consisted of 100 Hz trains of 0.5 s once every second for 60 s. Fatigue was electrophysiologically determined as the force reduction relative to the initial force. Force was normalized for muscle weight. As shown in FIG. 17, oleuropein (standardized at 40%, from olive leaf extract, OLE) increases resistance to fatigue in vivo in young adult mice after chronic treatment.

To test the chronic effect of oleuropein on running performance in an endurance treadmill test in healthy adult mice, 3-month-old mice were supplemented with olive leaf extract, standardised for its oleuropein content (20 mg/kg/day oleuropein) for 4 weeks. Control mice were treated with chow diet. Mice were acclimated to and trained on a 10° uphill LE8700 treadmill (Harvard apparatus) for 2 days. On day 1, mice ran for 5 min at 8 m/min and on day 2 mice ran for 5 min at 8 m/min followed by another 5 min at 10 m/min. On day 3, mice were subjected to a single bout of running starting at the speed of 10 m/min. Ten minutes later, the treadmill speed was increased at a rate of 2 m/min every 5 min until mice were exhausted. Exhaustion was defined as the point at which mice spent more than 5 s on the electric shocker without attempting to resume running. Total running time was recorded for each mouse.

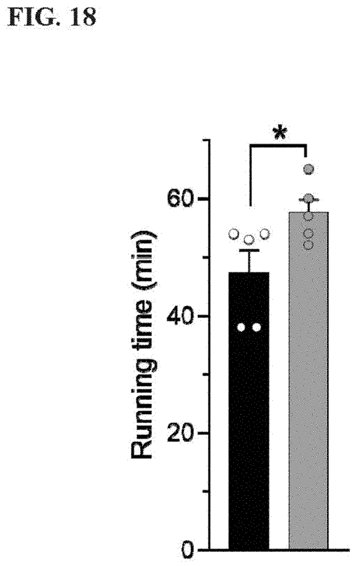

FIG. 18 is a graph showing that oleuropein (standardized at 40% from olive leaf extract) increases running capacity in vivo in young adult mice after chronic treatment. To test the chronic effect of oleuropein on running performance in an endurance treadmill test in old mice, 20-month-old mice were supplemented with olive leaf extract, standardised for its oleuropein content (20 mg/kg/day oleuropein) for 4 weeks. Control mice were treated with chow diet. Experiment were performed as described above, for young adult mice.

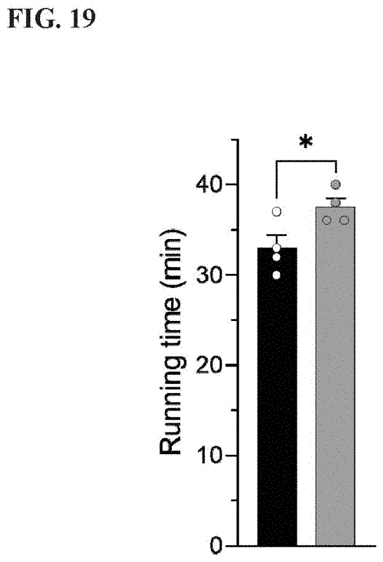

FIG. 19 is a graph showing that oleuropein (standardized at 40% from olive leaf extract) increases running capacity in vivo in old mice after chronic treatment.

### REFERENCES

Alvarez, J., & Montero, M. (2002). Measuring [Ca2+] in the endoplasmic reticulum with aequorin. Cell Calcium, 32(5-6), 251-260.
Montero, M., Lobaton, C. D., Hernandez-Sanmiguel, E., Santodomingo, J., Vay, L., Moreno, A., & Alvarez, J. (2004). Direct activation of the mitochondrial calcium uniporter by natural plant flavonoids. Biochem J, 384(Pt 1), 19-24. doi:10.1042/BJ20040990

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of preventing or treating muscle fatigue from exercise and/or for resistance to muscle fatigue from exercise, the method comprising orally administering an effective amount of at least one of oleuropein or metabolite thereof to an individual before, during and/or after the exercise.

2. The method of claim 1, wherein the metabolite of oleuropein is selected from the group consisting of oleuropein aglycone, hydroxytyrosol, homovanillyl alcohol, isohomovanillyl alcohol, glucuronidated forms thereof, sulfated forms thereof, derivatives thereof, and mixtures thereof.

3. The method of claim 1 or claim 2, wherein the effective amount of at least one of oleuropein or metabolite thereof is administered in a composition selected from the group consisting of food compositions, dietary supplements, nutritional compositions, nutraceuticals, beverages, powdered nutritional products to be reconstituted in water or milk before consumption, food additives, medicaments, drinks, petfood, and combinations thereof.

4. The method of any of claims 1 to 3, wherein the effective amount of at least one of oleuropein or metabolite thereof is administered in a composition further comprising calcium.

5. The method of any of claims 1 to 4, wherein the effective amount of at least one of oleuropein or metabolite thereof is administered in a food product further comprising a component selected from the group consisting of protein, carbohydrate, fat and mixtures thereof.

6. The method of any of claims 1 to 5, wherein the exercise is one or more of 1) resistance exercise, 2) anaerobic or repeated sprint-type exercise, or 3) endurance exercise.

7. A unit dosage form comprising at least one of oleuropein or metabolite thereof in an amount effective for administration of the unit dosage form before, during and/or after exercise to thereby prevent or treat muscle fatigue from the exercise and/or for resistance to muscle fatigue from exercise.

8. The unit dosage form of claim 7, consisting essentially of the at least one of oleuropein or metabolite thereof.

9. The unit dosage form of claim 7 or claim 8, consisting of an excipient and the at least one of oleuropein or metabolite thereof.

10. The unit dosage form of any of claims 7 to 9, consisting essentially of calcium and the at least one of oleuropein or metabolite thereof.

11. The unit dosage form of any of claims 7 to 10, consisting of an excipient, calcium, and the at least one of oleuropein or metabolite thereof.

12. The unit dosage form of any of claims 7 to 11, wherein the exercise is one or more of 1) resistance exercise, 2) anaerobic or repeated sprint-type exercise, or 3) endurance exercise.

13. A method of making a composition for preventing or treating muscle fatigue from exercise and/or for resistance to muscle fatigue from exercise, the method comprising adding an effective amount of at least one of oleuropein or metabolite thereof to at least one ingredient selected from the group consisting of protein, carbohydrate, and fat.

14. The method of claim 13 further comprising adding to the at least one ingredient a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

15. The method of claim 13 or claim 14 further comprising adding calcium to the at least one ingredient.

16. The method of any of claims 13 to 15, wherein the exercise is one or more of 1) resistance exercise, 2) anaerobic or repeated sprint-type exercise, or 3) endurance exercise.
